(19) United States
(12) Patent Application Publication
(10) Pub. No.: US 2007/0042353 A1
(43) Pub. Date: Feb. 22, 2007

(54) HCV polymerase suitable for crystal structure analysis and method for using the enzyme

(75) Inventors: Hideo Ago, Osaka (JP); Masashi Miyano, Tokyo (JP); Tsuyoshi Adachi, Osaka (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Japan Tobacco Inc.

(21) Appl. No.: 11/033,976

(22) Filed: Jan. 13, 2005

Related U.S. Application Data
(63) Continuation of application No. 09/608,713, filed on Jun. 30, 2000, now abandoned.

(30) Foreign Application Priority Data
Jul. 2, 1999 (JP) 11-188630
Jul. 7, 1999 (JP) 11-192488

(51) Int. Cl. C12Q 1/70; C12N 9/22; G06F 19/00

(52) U.S. Cl. 435/5; 702/19; 435/199

(57) ABSTRACT

An HCV polymerase suitable for crystal structural analysis and a method for using the enzyme are provided. The HCV polymerase suitable for crystal structural analysis and/or comprising high HCV polymerase activity can be used for the three-dimensional structural analysis, and for rational identification of HCV polymerase inhibitors by computers. The enzyme can also be used for efficiently evaluating the HCV polymerase-inhibitory activity. The evaluation can be more efficiently performed by combining identification by computers.

### FIELD OF THE INVENTION

The present invention relates to a polypeptide having the hepatitis C virus (HCV) polymerase activity, suitable for crystal structure analysis, and effective for evaluating the HCV polymerase activity and the use of the polypeptide.

More specifically, the present invention relates to a polypeptide having the HCV polymerase activity, which is obtained in the form of crystals suitable for crystal structure analysis and its crystals, as well as a DNA encoding the polypeptide. The present invention also relates to: (a) a method for determining structural coordinates for a cocomplex or a variant of the polypeptide (NS5B), (b) a method for identifying HCV polymerase inhibitors from the complementarity of a test compound with an active site and/or RNA binding cleft of the polypeptide, and (c) an HCV polymerase inhibitor obtained by the methods.

Moreover, the present invention relates to a method for identifying HCV polymerase inhibitors using the polypeptide that shows the polymerase activity higher than the wild-type HCV polymerase.

### BACKGROUND OF THE INVENTION

Hepatitis C is a grave problem as it infects by blood transfusion and so on, and more than half of the cases become chronic with the high probability of progressing into cirrhosis and hepatoma. A cause of hepatitis C is known to be hepatitis C virus (HCV) and its gene was cloned in 1989 by the immunoscreening method using plasma of the chimpanzee infected with the human plasma (Science, 244, 359-362, 1989).

Hepatitis C virus is a plus strand RNA virus with an envelope and comprises the RNA encoding a protein consisting of 3010 amino acids. A precursor protein biosynthesized from the RNA in a host is processed into a structural protein forming viral particles (a core protein and two envelope proteins) and a non-structural protein (NS2, NS3, NS4A, NS4B, NS5A, NS5B) by a cellular signalase and a protease encoded by the RNA of the virus itself. It has been considered that NS2 and NS3 retain the protease activity and are necessary enzymes for processing a precursor protein, and the helicase of NS3 and RNA-dependent RNA polymerase of NS5 are essential for viral replication.

At present, interferon α and interferon β are used for treating hepatitis C, however, they are less or no effective for many patients. A more effective drug for the treatment is thus needed. Developing novel HCV inhibitors is underway, and attention is focused on studies on the inhibitors by targeting proteins specific for HCV, such as protease, helicase, RNA-dependent RNA polymerase.

An inhibitor for viral proliferation is generally screened by measuring activity of inhibiting viral proliferation in vitro or in vivo. As to HCV, however, techniques for effecting the viral proliferation in vitro has not been established yet. Moreover, the screening of the viral proliferation of HCV in hosts is difficult because the virus only infects cells of human and chimpanzee.

Therefore, in development of anti-HCV drugs, developing an inhibitor by targeting specific coding proteins essential for the HCV proliferation is of great significance and the efficient assay method is desired.

In developing inhibitors for enzyme activity, molecular designing of inhibitors has been attempted by computers based on three-dimensional structure of enzymes to enhance the screening efficiency. In this methodology, various candidate compounds are designed and identified by tentatively evaluating the inhibitory activity against the enzyme activity of the candidate compounds by computer, considering the three-dimensional structures of various candidate compounds and physical properties of the molecule. The inhibitors can be identified more efficiently by combining designing and evaluation of the inhibitors using the three-dimensional structures of these enzymes, and evaluation of the enzyme activity in actually synthesized compounds.

In order to design molecules of inhibitors by computers, three-dimensional structure of an enzyme must be revealed. Three-dimensional structure of the enzyme can be clarified by X-ray crystal structure analysis. For example, the crystal structures of HIV reverse transcriptase (Nature Structural Biology, 2, 293-302, 1995; Structure, 3, 365-379, 1995), interleukin-1β transformation enzyme (WO95/35367), protease of cytomegalovirus (WO97/42311), HCV helicase (WO99/09148), etc., have been analyzed.

An enzyme is a macromolecular compound, and its structure is complicated. X-ray structure analysis requires crystallization of the enzyme to strengthen diffraction intensity obtained by X-ray radiation. The structure cannot be fully analyzed unless the enzyme is stably crystallized and the crystals are produced in a large amount. Thanks to the development of the recombinant technique, a large amount of enzymes can be homogeneously and highly purified. However, it is difficult to obtain enzyme crystals suitable for X-ray analysis, and if at all, the structure cannot be completely analyzed in many cases. For example, the reported crystal structure of poliovirus RNA-dependent RNA polymerase (Structure 5, 1109-1122, 1997) is not complete, and only some parts have been analyzed, presumably because partial structures in the crystallized enzyme are disordered, and the protein has no stable structure.

Based on such a background, the results of the studies on crystal structure analysis of the HCV polymerase have been recently published. In Nature Structural Biology, 6 (10), 937-43, (Oct., 1999), the three-dimensional structure of NS5B was analyzed by adding hexahistidine tag to the N-terminus of NS5B consisting of 570 amino acids and analyzing it with X-ray. In Proc. Natl. Acad. Sci. USA, 96 (23), 13034-39 (Nov. 1999), the sequence of 531 amino acids of NS5B was disclosed and the three-dimensional structure of NS5B was analyzed in the same manner. However, both of these references were published after the priority date of the present application. Moreover, the references did not describe that the HCV polymerase obtained showed activity higher than the activity of the wild type; the references neither disclose nor suggest usefulness of the HCV polymerase for the actual enzyme activity evaluation.

WO99/43792 discloses a novel HCV polymerase useful for evaluating enzyme activity, which is NS5B consisting of 570 amino acids and its variant having the glutathione S-transferase tag sequence at the N-terminus.

However, the publication did not disclose or suggest that the polymerase was useful for X-ray structure analysis or suitable for crystallization. There was no description or suggestion about a method for identifying HCV polymerase inhibitors using the three-dimensional structure.

It has been implied that the C-terminal structure of the HCV polymerase may effect self-inhibition against the replication of RNA (Structure, 7 (11), 1417-26, (Nov. 1999)). In fact, the C-terminus-deficient variant has reportedly high RNA-dependent RNA polymerase activity (Journal of Virology, 73, 1649-54, Feb. 1999, and Journal of General Virology, 81, 759-767, 2000). In the former reference, however, NS5B₅₃₆ (NS5B consisting of 536 amino acids in which the C-terminus of the full length NS5B is truncated, same hereafter) and NS5B₅₂₈, showed only slightly higher activity compared with NS5B₅₇₀ (about 1.3 to 1.4 times). In the latter, activity of NS5B₅₉₁ (the full length NS5B) was compared with only that of NS5B₅₇₀. These references demonstrate that the truncated NS5B retained the polymerase activity, but do not propose any methods for evaluating inhibition for the HCV polymerase activity more efficiently by exploiting the high polymerase activity. Moreover, these references do not suggest any method for identifying compounds having the HCV polymerase-inhibitory activity more efficiently in combination with a method for identifying inhibitors based on the three-dimensional structure of the HCV polymerase.

### SUMMARY OF THE INVENTION

An objective of the present invention is to provide a polypeptide having HCV polymerase activity, which is obtained in the form of crystals suitable for crystal structure analysis, the crystals, and a DNA encoding the polypeptide. Another objective of the present invention is to provide, (a) a method for determining structural coordinates of a cocomplex and a variant of the polypeptide, (b) a method for identifying HCV polymerase inhibitors based on the complementarity of a test compound with the active site and/or the RNA binding cleft of the polypeptide, and using the structural coordinate, and (c) HCV polymerase inhibitors obtained by the above methods.

Still another objective of the present invention is to provide a polypeptide having polymerase activity higher than that of the wild-type HCV polymerase, a method for inhibiting the HCV polymerase using the polypeptide, and a method for identifying HCV polymerase inhibitors using the methods.

The present inventors extensively studied to find polypeptides having the HCV polymerase activity, which can be obtained in the form of crystals suitable for crystal structure analysis. The inventors discovered a desired polypeptide and successfully clarified its crystal structure to complete the present invention.

Specifically, the present invention is described in (1) to (16) below.

(1) A polypeptide derived from HCV polymerase NS5B having an HCV polymerase activity and consisting of an amino acid sequence X-Y, wherein X is a consecutive amino acid sequence which is a portion of the NS5B, an N-terminal amino acid of X is the amino acid residue 1 (Ser) of the NS5B, and a C-terminal amino acid residue of X is any one of amino acid residues 531 (Lys) to 570 (Arg) of the NS5B; and wherein Y is a carboxyl group or an amino acid sequence which is not derived from NS5B; and one or more amino acids in the amino acid sequence of X may be modified, and methionine residues in the amino acid sequence of X may be replaced by selenomethionoine residues.

(2) The polypeptide of (1), wherein the C-terminal amino acid residue of X is any one of amino acid residues 536 (Leu) to 552 (Val) of the NS5B.

(3) The polypeptide of (2), wherein the C-terminal amino acid residue of X is any one of amino acid residues 536 (Leu) to 544 (Gln) of the NS5B.

(4) The polypeptide of (2), wherein the C-terminal amino acid residue of X is any one of amino acid residues 531 (Lys) to 544 (Gln) of the NS5B.

(5) The polypeptides of any one of (1) to (4), wherein methionine residues in the amino acid sequence of X are replaced by selenomethionine residues.

(6) The polypeptides of any one of (1) to (5), wherein Y is an amino acid sequence not derived from NS5B, and said amino acid sequence is suitable for a column purification.

(7) The polypeptides of any one of (1) to (6), wherein the NS5B comprises an amino acid sequence of SEQ ID NO: 1.

(8) The polypeptide of (1), wherein said polypeptide is identified by an three-dimensional structural coordinates shown in Tables 2 or 3.

(9) A crystal comprising the polypeptide of any one of (1) to (8).

(10) A DNA encoding the polypeptide of any one of (1) to (8).

(11) A method for determining a three-dimensional structural coordinates of a cocomplex or a variant of HCV polymerase NS5B by the molecular replacement method using a three-dimensional structure coordinate of said NS5B.

(12) A method for designing or identifying HCV polymerase inhibitors, which comprises determining the complementarity of a test compound with an active site and/or RNA binding cleft of a polypeptide using the three-dimensional structural coordinate of said polypeptide or its part and the three dimensional structural coordinate of the test compound, wherein said polypeptide is derived from the HCV polymerase NS5B having an HCV polymerase activity and consisting of an amino acid sequence X-Y, wherein X is a consecutive amino acid sequence which is a portion of the NS5B, an N-terminal amino acid of X is amino acid residue 1 (Ser) of the NS5B, a C-terminal amino acid residue of X is any one of amino acid residues 531 (Lys) to 570 (Arg) of the NS5B: and wherein Y is a carboxyl group or another amino acid sequence which is not derived from NS5B; and one or more amino acids in X may be modified, and methionine residues in the amino acid sequence of X may be replaced by selenomethionine residues.

(13) A method for designing or identifying HCV polymerase inhibitors, which comprises the steps of:

(a) determining the complementarity of a test compound with an active site and/or RNA binding cleft of the a polypeptide using a three-dimensional structural coordinate of said polypeptide or its part and a three-dimensional structural coordinate of said test compound, wherein said polypeptide is derived from the HCV polymerase NS5B having an HCV polymerase activity and consisting of an amino acid sequence X-Y, wherein X is a consecutive amino acid sequence which is a portion of the NS5B, an N-terminal amino acid of X is the amino acid residue 1 (Ser) of the NS5B, a C-terminal amino acid residue of X is any one of amino acid residues 531 (Lys) to 570 (Arg) of the NS5B; and wherein Y is a carboxyl group of another amino acid sequence which is not derived from NS5B; and one or more amino acids in X may be modified, and methionine residues in the amino acid sequence of X may be replaced by selenomethionin residues.

(b) determining HCV polymerase-inhibitory activity of said test compound, and

(c) designing or determining HCV polymerase inhibitors using the complementarity data of said test compound determined in the above (a), and the inhibitory activity data obtained in the above (b).

(14) The method of any one of (11) to (13), wherein the three-dimensional structural coordinate of the polypeptide is any one of the three-dimensional structural coordinates shown in Table 2 or 3.

(15) A method for identifying HCV polymerase inhibitors, which comprises the steps of:

(a) obtaining a polypeptide, which is derived from the HCV polymerase NS5B has an HCV polymerase activity, and consisting of an amino acid sequence X′-Y, wherein X′ is a consecutive amino acid sequence which is a portion of the NS5B, an N-terminal amino acid of X′ is the amino acid residue 1 (Ser) of the NS5B, a C-terminal amino acid residue of X′ is any one of amino acid residues 531 (Lys) to 544 (Gln) of the NS5B; and wherein Y is a carboxyl group or another amino acid sequence which is not derived from NS5B; and one or more amino acids in X′ may be modified, and methionine residues in the amino acid sequence of X′ may be replaced by selenomethionin residues;

(b) determining the HCV polymerase activity of said polypeptide by reacting said polypeptide obtained in the above (a) with a template RNA and substrates in the presence of a test compound;

(c) determining the HCV polymerase activity of said polypeptide by reacting the polypeptide obtained in the above (a) with a template RNA and substrates in the absence of said test compound; and

(d) comparing the HCV polymerase activity of the above (b) with the HCV polymerase activity of the above (c).

(16) An HCV polymerase inhibitor, identified by the method in any one of (12) to (15).

(17) An HCV polymerase inhibitor that inhibits the HCV polymerase activity of HCV polymerase NS5B by acting the boundary between the Thumb and Palm domains of NS5B.

(18) The HCV polymerase inhibitor of (17), wherein said inhibitor is a polypeptide represented by the formula (I) or a pharmaceutically acceptable salf thereof:
Z¹-Z²-Z³-Leu-Z⁴-Z⁵-Trp-Phe-Z⁶ (I)
wherein Z¹ and Z⁶ each represent a hydrophilic group of an amino acid residue; Z² and Z³ each represent a single bond or an amino acid residue; and Z⁴ and Z⁵ each represent an amino acid residue.

The terms used herein have the following meanings.

“HCV” is an abbreviation for Hepatitis C Virus.

“HCV polymerase” means RNA-dependent RNA polymerase encoded by RNA of HCV. It means not only a specific sequence but also a polypeptide derived from HCV polymerase NS5B, including any specific HCV types, any genotypes, and any mutants having the polymerase activity.

The HCV polymerase includes a polypeptide derived from the HCV polymerase NS5B, which has a polymerase activity and consisting of an amino acid sequence X-Y, wherein X is a consecutive amino acid sequence which is a portion of the NS5B, an N-terminal amino acid of X is the amino acid residue 1 (Ser) of the NS5B, and a C-terminal amino acid residue of X is any one of the amino acid residues from 531 (Lys) to 570 (Arg) of the NS5B; and wherein Y is a carboxyl group or an amino acid sequence which is not derived from NS5B; and one or more amino acids in the amino acid sequence of X may be modified, and methionine residues in the amino acid sequence of X may be replaced by selenomethionoine residues.

Except as otherwise set forth herein, specific amino acid position(s) indicated in the present specification means the position(s) in NS5B amino acid sequence.

The amino acid sequence of SEQ ID NO: 1 is the HCV polymerase of HCV-BK strain belonging to HCV1b genotype, commonly observed worldwide, especially in Asia and Europe. Therefore it is a useful HCV polymerase in this invention.

“HCV polymerase NS5B” and “NS5B” mean nonstructural proteins encoded by the nonstructual. 5B (NS5B) region of the HCV RNA, which is located at the C-terminus of the viral protein (open reading frame) and possess RNA-dependent RNA polymerase activity. Herein, “HCV polymerase NS5B” and “NS5B” are synonyms with each other, and are not restricted to a specific wild-type NS5B.

NS5B is digested with a protease of the HCV itself. For example, in the case of the HCV-BK strain, NS5B is a polypeptide consisting of 59.1 amino acids from amino acid 2420 to the C-terminus in the viral protein (open reading frame).

A consecutive amino acid sequence 1-531 in NS5B is also designated as NS5B₅₃₁, and other consecutive amino acids are designated in the same manner.

“HCV polymerase activity” means the function of the above HCV n polymerase to amplify RNA.

“HCV polymerase-inhibitory activity” means the function to inhibit the above HCV polymerase activity.

“HCV polymerase inhibitor” means an agent having the activity of inhibiting the above HCV polymerase activity.

“Wild-type HCV polymerase” means full length RNA-dependent RNA polymerase encoded by the RNA of hepatitis C virus existing in nature.

“Native HCV polymerase” means the above HCV polymerase in which methionine is not replaced by selenomethionine.

“Substrate” means adenine, guanine, cytosine, uridine, and their ribonucleosides, preferably ribonucleoside triphosphates, ATP, GTP, CTP, and UTP.

“X is a consecutive amino acid sequence which is a portion of the NS5B, and an N-terminal amino acid of X is an amino acid residue 1 (Ser) of the NS5B, and a C-terminal amino acid residue of X is any one among amino acid residues 531 (Lys) to 570 (Arg)” specifically means all amino acid residues designated by consecutive amino acid residues 1 to 531, consecutive amino acid residues 1 to 532, consecutive amino acid residues 1 to 533, a consecutive amino acid residues 1 to 570, and so on.

To obtain crystals suitable for crystal structure analysis, a C-terminal amino acid residue of X is preferably any one among amino acid residues 531 to 570 of NS5B. As for specific polypeptide, NS5B₅₃₁, NS5B₅₃₆, NS5B₅₄₄, or NS5B₅₇₀ are preferable, NS5B₅₃₆, NS5B₅₄₄, or NS5B₅₇₀ are more preferable, and NS5B₅₄₄ and NS5B₅₇₀ are still more preferable. NS5B₅₄₄ is the most preferable because its crystals are of high quality.

In order to obtain NS5B having activity higher than the wild-type HCV polymerase, suitable for the enzyme assay, a C-terminal amino acid residue of X is preferably any amino acid residue among amino acid residues 531 to 554. Specifically, NS5B₅₃₁, NS5B₅₃₆, or NS5B₅₄₄ are preferable, and NS5B₅₄₄ is more preferable.

X′ means the same amino acid residue as X except that a C-terminal amino acid residue is any amino acid residue among amino add residues 531 to 554 of NS5B.

“One or more amino acids in the amino acid sequence of X may be modified” means that 1 to 20, preferably 1 to 10, and more preferably 1 to 5 amino acids may be modified in the amino acid sequence of X. The modification includes natural or artificial deletion, replacement, and addition, and modification of the N- and/or C-terminal amino acid sequences.

“Selenomethionine” means a substituted methionine in which a sulfur atom is replaced by a selenium atom.

“Amino acid sequence suitable for column purification” means any amino acid sequence that can specifically bind to a carrier used for an affinity column used for purification of the polypeptide. Such a sequence is preferably a sequence which can be easily cleaved after the purification with the affinity column and/or does not prevent crystallization of the polypeptide, and includes, for example, the histidine tag sequence, and the glutathione S-trasferase tag sequence. The histidine tag needs four or more histidines, and specifically includes—Gly-Ser-His-His-His-His-His-His,-Gly-Ser-His-His-Asp-His-His-His, etc.

In the formula (I), Z² is preferably a single bond, and Z¹, Z³, Z⁴, Z⁶, and Z⁶ are preferably any of amino acid residues.

Amino acid residues for Z², Z³, Z⁴, and Z⁵, are preferably hydrophilic amino acid residues, including Gly, Ser, Thr, Cys, Thr, Asn, Gln, Lys, His, Arg, Asp, and Glu. Z³ is preferably Asp, Z⁴ is preferably Ser, and Z⁵ is preferably Gly.

An amino acid residue for Z¹ is preferably Leu or a hydrophilic amino acid residue, which includes the same examples as for Z², Z³, Z⁴, and Z⁵. Z⁶ is more preferably Leu or Lys, and still more preferably Lys.

An amino acid residue for Z⁶ is preferably Val or a hydrophilic amino acid group, which includes the same examples as for Z², Z³, Z′, and Z⁵. Z⁶ is more preferably Val, Thr, Arg or Lys, and still more preferably Lys.

“Hydrophilic group” includes a hydroxyl group, a carboxyl group, an amino group, a dimethylamino group, a mercapto group, and so on, and also include a carboxyl group contained in a phenylalanine residue at the C-terminus of the polypeptide represented by the formula (I), and an amino group contained in the N-terminal residue of said polypeptide. A hydrophilic group for Z¹ is preferably an amino group, and that for Z⁶ is preferably a carboxyl group.

Examples of the polypeptide represented by the formula (I) include:
Lys-Asp-Leu-Ser-Gly-Trp-Phe-Lys;

Lys-Lys-Asp-Leu-Ser-Gly-Trp-Phe-Lys;
Lys-Asp-Leu-Ser-Gly-Trp-Phe-Val;
Leu-Asp-Leu-Ser-Gly-Trp-Phe-Lys;
Leu-Asp-Leu-Ser-Gly-Trp-Phe-Val;
   Asp-Leu-Ser-Gly-Trp-Phe-Val;
   Asp-Leu-Ser-Gly-Trp-Phe;
      Leu-Ser-Gly-Trp-Phe-Val;
      Leu-Ser-Gly-Trp-Phe;
      Leu-Ser-Gly-Trp-Phe-Lys;
   Lys-Leu-Ser-Gly-Trp-Phe;
      Leu-Gly-Gly-Trp-Phe;
      Leu-Ser-Asp-Trp-Phe; etc.

The polypeptide is preferably
Lys-Asp-Leu-Ser-Gly-Trp-Phe-Lys and
Leu-Asp-Leu-Ser-Gly-Trp-Phe-Val,
   and more preferably
Lys-Asp-Leu-Ser-Gly-Trp-Phe-Lys.

“Three-dimensional structural coordinate” and “structural coordinate” of the HCV polymerase NS5B are synonymously used, and the structural coordinate includes “structural coordinate substantially equivalent” to “structural coordinate” of the NS5B.

“Structural coordinate” is a mathematical coordinate obtained by converting diffraction intensity at each diffraction point obtained by X-ray diffraction by electrons contained in atoms of the NS5B in the crystal form, into a numerical value, and analyzing the result. It presents locations of atoms in the NS5B expressed as a three-dimensional coordinate. Specifically, examples are the structural coordinates shown in Tables 2 and 3 of Example 2.

“Substantially equivalent structural coordinate” means a derivative structural coordinate generated as a result of artificially processing the structural coordinate of the NS5B or its part by computers or the like means. When the derivative structural coordinate of the NS5B is overlapped on the structural coordinate shown in Tables 2 or 3 so as to fit the locations of the corresponding atoms, residual mean square deviation is preferably within ±0.5 Å or less, and more preferably, ±0.2 Å or less from an original atom. A structural coordinate is preferably that of NS5B having the polymerase activity. Two structural coordinates show the identical three-dimensional structure when the locations of the corresponding atoms included in the structural coordinates can be overlapped, even if the numerical values of the coordinates indicating the locations of the atoms are different.

“To identify” means not only determining one, but selecting the less from the more.

“Molecular replacement method” is a method for determining the crystal structure of a protein whose structure is unknown based on the structure of a known protein with the same function as an initial model. Specific procedures are described in Experimental Chemistry Course 10, Diffraction, Japanese Society of Chemistry, 260-263 (1992), or Methods in Enzymology, 115, 55-77 (1985), edited by M. G. Rossman.

“Cocomplex” means a complex formed by HCV polymerase and a compound having or expectedly having the HCV polymerase-inhibitory activity, and includes a complex comprising RNA strands, substrates or a metal essential for expression of the HCV polymerase activity, for example, manganese, magnesium, etc. Cocomplexes include are those formed by cocrystals, and those formed by soaking the HCV polymerase crystals in a solution containing the compound having or expectedly having the HCV polymerase-inhibitory activity.

“Active site” means (1) the region of the HCV polymerase in which a template RNA is replicated, formed by Asp at positions 220, 318 and 319, Lys 144, and Arg 158 in the amino acid sequence of the HCV polymerase, and/or (2) a hydrophilic shallow hollow formed by Ser 282, Thr 287, and Asn 291.

“RNA binding cleft” means a portion of the HCV polymerase, including an active site, and the inner space formed by the following Fingers, Palm and Thumb domains. It is a site that can be a target for identifying HCV polymerase inhibitors, including a space where a template RNA is incorporated when RNA is replicated. “RNA binding cleft” used herein differs from an active site.

Specific examples of the “RNA binding cleft” includes “inner space of the Palm domain” and “boundary site between the Thumb and Palm domains”, other than the above “active site.”

“Inner space of the Palm domain” is not an RNA replication site, but a space generated between the HCV polymerase and a template RNA when RNA is replicated using RNA of HCV as a template, and formed by the regions of amino acid residues 197 to 223, 310 to 325, and 348 to 366. These regions may be shifted 1 to 20, preferably 1 to 10, and more preferably 1 to 5 amino acids, to the N- or C-terminal side.

“Boundary site between the Thumb and Palm domains” is not an RNA replication site, but a site to which the C-terminus of NS5B₅₇₀ binds, and which can be a target for identifying HCV polymerase inhibitors, comprising the hydrophobic surface existing at the boundary between the Thumb and Palm domains described below. Specifically, the site is formed by amino acid Ser 196, Pro 197, Ile 413, Met 414, Ile 447, Tyr 448, Tyr 452, Ile 454, Ile 462, and Leu 466 in the amino acid sequence of the HCV polymerase or a part of it.

A site involved in “RNA binding cleft” includes Lys 90, 98, 106, and 172 and Arg 168 in the Holder domain, and Arg 465 in the Thumb domain, which have an important role in binding of RNA strands.

A preferable site that can be a target for identifying HCV polymerase inhibitors includes “active site” and “boundary site between Thumb and Palm domains.”

“A part of a structural coordinate” means the structural coordinate including all or some of structures shown in the above “active site” and “RNA binding cleft” among the structural coordinates of NS5B.

“Complementarity of a test compound with an active site and/or RNA binding cleft of the polypeptide” is determined by calculating the condition under which the test compound conformationally or physically interacts with an active site and/or RNA binding cleft, and converting binding stability of the test compound to the site into a numerical value or visualizing the binding stability. Especially, it is preferable to obtain the complementarity of the surface structure of the test compound with the surface structure of the polypeptide by calculating conformational or static complementarity.

Complementarity can be compared between unknown compounds A and B, between a known compound A with a known enzyme-inhibitory activity and an unknown compound A, between known compounds A and B, and between any compounds.

“Acting on the boundary site between the Thumb and Palm domains” means that an HCV polymerase inhibitor binds to said site of HCV polymerase NS5B physically, chemically, statically, or in a similar manner, but is not limited to these modes of action.

Any patents, patent applications, and publications cited herein are incorporated by reference.

### BRIEF DESCRIPTION OF THE DRAWINGS

FIG. 1 shows the ribbon model of the three-dimensional structure obtained by visualizing the structural coordinate of the HCV polymerase (NS5B₅₇₀) using the program software RasMol.

FIG. 2 schematically shows the three-dimensional structure of the HCV polymerase (NS5B₅₇₀). The α helices and β sheets are indicated sequentially in alphabetical and numerical order, respectively.

FIG. 3 compares amino acid sequences of HCV polymerase, poliovirus polymerase, and HIV reverse transcriptase.

FIG. 4 shows the three-dimensional structure of the HCV polymerase NS5B₅₇₀, emphasizing the polypeptide region at positions 547 to 556. The right figure corresponds to the left figure rotated 90 degree upward.

### DETAILED DESCRIPTION OF THE INVENTION

The present invention is illustrated in detail below.

i) HCV Polymerase Suitable for Crystal Structure Analysis and its Gene

The HCV polymerase suitable for crystal structure analysis of the present invention can be prepared by the standard methods of recombinant techniques.

For example, a DNA encoding a polypeptide derived from the HCV polymerase NS5B is inserted into a vector. The polypeptide has the HCV polymerase activity and comprises an amino acid sequence X-Y, in which X is a consecutive amino acid sequence which is a part of NS5B, an N-terminal amino acid of X is the amino acid residue 1 (Ser) of NS5B, and a C terminal amino acid residue of X is any one of the amino acid residues from 531 (Lys) to 570 (Arg) of NS5B; Y is a carboxyl group or an amino acid sequence which is not derived from NS5B; and one or more amino acids in the amino acid sequence of X may be modified, and methionine of the amino acid sequence of X may be replaced by selenomethionoine. The vector is used to transform E. coli, and the resulting transformants are cultured to isolate the polypeptide.

The HCV polymerase “suitable for crystal structure analysis” should be easily purified.

One embodiment of the HCV polymerase is a variant of the HCV polymerase in which a part of the amino acids are replaced by amino acids suitable for column purification. The replaced sequence makes the purification of the polypeptide easy and its large quantity production possible. An amino acid sequence of the HCV polymerase, suitable for crystallization, is preferably a sequence in which the amino acids suitable for column purification can be readily cleaved after column purification and/or which does not prevent crystallization of the HCV polymerase. Purification without using a surfactant is very preferable in crystallization after the purification.

Furthermore, the HCV polymerase “suitable for crystal structure analysis” should be easily crystallized, made into crystals with high quality, which are hardly degraded. “Being easily crystallized” means that manipulation for crystallization is easy, conditions for crystallization are simple, and that crystals can be obtained with no or little influence by a small difference of conditions for crystallization, for example, temperature and kinds, concentration, pH of a solvent, and so on. “Crystals with high quality” means less lattice fault and larger crystals. “Being hardly degraded” means, for example, that the crystals are not easily dissolved according to the change of, for example, temperature and kinds, concentration, and pH of a solvent.

The crystals should have smaller fluctuation in a whole molecule or a part of the molecule of the crystallized polymerase.

The crystals of the obtained HCV polymerase can be grown, for example, by vapor diffusion, to be used for crystal structure analysis.

A cocrystal comprising the polymerase and a compound having the HCV polymerase-inhibitory activity can be used for crystal structure analysis of the cocomplex. Crystals prepared by soaking the polymerase crystal in a solution of the compound can also be used for crystal structure analysis of the cocomplex.

It is generally known that when an amino acid sequence of a physiologically active protein is slightly modified, for example, by deletion or substitution of one or more amino acids in the amino acid sequence, or addition of one or more amino acids to the amino acid sequence, the physiological activity of the protein may be retained. Not only artificial manipulation but also spontaneous mutation may retain the activity.

A polypeptide modified by deletion, substitution, or addition of amino acid(s), can be prepared by, for example, subjecting the gene encoding the polypeptide to the site-directed mutagenesis that is known in the art (for example, Nucl. Acid Research, 10 (20), 6487-6500, 1992). For example, the mutation can be performed by using synthetic oligonucleotide primers complementary to the DNA at a site where the corresponding polypeptide is to be modified.

In addition to the site-directed mutagenesis, known modification methods include a method in which a gene is treated with mutagen, or a method in which a gene is cleaved with a restriction enzyme, and a selected gene fragment is removed, added or replaced, and ligated.

A variant may include a conservatively substituted sequence. This indicates that a specific amino acid residue may be substituted by a residue with similar physiochemical properties. Unrestricted examples of the conservative substitution include the substitution among amino acid residues with an aliphatic chain, such as the substitution among Ile, Val, Leu, or Ala, or the substitution between Lys and Arg which are basic amino acids having a polar group.

The present invention provides a DNA encoding the HCV polymerase, which is a DNA encoding the polypeptide derived from the HCV polymerase NS5B, having the HCV polymerase activity and comprising the amino acid sequence X-Y, in which X is a consecutive amino acid sequence which is a part of NS5B, an N terminal amino acid of X is the amino acid residue 1 (Ser) of NS5B, and a C terminal amino acid residue of X is any one of the amino acid residues from 531 (Lys) to 570 (Arg) of NS5B; Y is a carboxyl group or an amino acid sequence which is not derived from NS5B; and one or more amino acids in the amino acid sequence of X may be modified, and methionine in the amino acid sequence of X may be replaced by selenomethionoine.

Since there are more than one codons which encode one amino acid, any DNA having a nucleotide sequence can be used as long as a desired amino acid sequence can be obtained. Therefore, the DNA of the present invention includes not only a DNA encoding an amino acid sequence of, for example, SEQ ID NO: 1, but also DNAs comprising any combinations of codons, which encode desired amino acid sequences.

ii) Crystal Structure Analysis of the HCV Polymerase

An enzyme such as the HCV polymerase, has the complicated molecular structure, thus three-dimensional structures of its crystals and the molecule can be identified by analyzing the crystals with X-ray. The crystal structure analysis can be performed by, for example, the molecular replacement method, the multiple wavelength anomalous dispersion method, the multiple heavy atom isomorphous replacement method, and so on.

Crystal Structure Analysis by the Multiple Heavy Atom Isomorphous Replacement Method

The multiple heavy atom isomorphous replacement method is a method for analyzing the three-dimensional structure of a protein, comprising measuring X-ray diffraction intensity of native protein crystals to which a heavy atom/s is/are attached, and the native protein crystals to which a heavy atom is not introduced (a native protein), comparing the diffraction intensity data, and determining a phase angle for each diffraction for the crystals (Experimental Chemistry Course 10, Diffraction, Japanese Society of Chemistry, 253-260, 1992).

To analyze the crystal structure of the HCV polymerase by the multiple heavy atom isomorphous replacement method, a desired HCV polymerase is produced and purified by the above method.

The HCV polymerase in which a sulfur of Met was replaced with selenium was isolated and purified by producing the HCV polymerase by the above method in the medium in which selenomethionine was added in place of methionine (hereafter referred to as the selenomethionine HCV polymerase or the selenomethionine heavy atom substitution product).

Herein, the HCV polymerase obtained by the addition of Met may be expressed as a native HCV polymerase to distinguish from the selenomethionine HCV polymerase.

The crystals of the native HCV polymerase and the selenomethionine HCV polymerase can be obtained by vapor diffusion. A heavy atom substitution products of the native HCV polymerase can be prepared by soaking the crystals of the native and the native HCV polymerase in solutions containing platinum, uranium, and osmium. A structural coordinate can be determined by measuring diffraction intensity for the crystals of the obtained heavy atom substitution products and calculating the phase angle by the multiple heavy atom isomorphous replacement method.

According to the principle of the multiple heavy atom isomorphous replacement method, there is the relationship for each reflection of a structural factor from the crystals with a heavy atom (FPH), a structural factor of the native crystal data (FP), and contribution of the introduced heavy atom (FH):
FPH=FP+FH.

|FPH| and |FP| are the numerical values obtained from the experiment. From these data, the location of the heavy atom can be determined, and |FH| can be caluculated to determine the phase angle of each reflection. Subsequently, the structure of the HCV polymerase can be determined by obtaining the electron density.

These calculation can be performed using program software DENZO, Shelx, MLPHARE, SHARP, DM, O, etc.

It is known that in general even if a structural coordinate for the location of each atom is changed to some extent on a computer, the structure does not largely change and the protein activity is not inactivated. Therefore, the structure coordinate essentially equivalent to that for the HCV polymerase of the present invention includes derivative coordinates prepared by artificially processing the coordinate of the HCV polymerase. Such derivative coordinates preferably show residual mean square deviation within the range of ±0.5 Å or less, and more preferably within the range of +0.2 Å or less, when the derivative coordinate is overlapped on the original structural coordinate so as to fit the locations of the atoms.

iii) Determination of “Active Site” and “RNA Binding Deft” of the HCV Polymerase

The active site of the HCV polymerase can be identified or deduced from the three-dimensional structure obtained by the crystal structure analysis of the HCV polymerase and the amino acid sequence. For identification and deduction, the amino acid sequence and the three-dimensional structure of a known polypeptide with the similar function can be referred.

From the obtained three-dimensional structure, a site which can be a target for inhibition of the HCV polymerase activity can be estimated in addition to the active site. The coordinate for the HCV polymerase obtained as a result of the structure analysis can be used, for example, for the following purposes:

(a) analysis of the crystal structure of the HCV polymerase variant;

(b) analysis of the crystal structure of the cocomplex composed of the HCV polymerase and the inhibitor; and,

(c) evaluation of the complementarity of a test compound with the active site and/or the RNA binding deft of the HCV polymerase.

iv) Crystal Structure Analysis of a Variant or a Cocomplex of the HCV Polymerase

Based on the structural coordinates for the HCV polymerase shown in Tables 2 and 3, the crystal structure of a variant or a cocomplex of HCV polymerase can be determined. The structural coordinate for the cocomplex can be important information for improving the quality of designing/evaluating a compound having the complementarity with the active site and/or the RNA binding cleft of the HCV polymerase.

In the molecular replacement method, rotational function is calculated from the crystal diffraction intensity data of the variant or the cocomplex of the HCV polymerase to determine the orientation of the molecule, and the location of the molecule is determined by calculating the translational function (Acta Crystallogr., 23, 544, 1967).

This method can be performed by using program software Amore of CCP4, Almn of CCP4 (Council for the Central Laboratory of the Research Councils), etc.

v) Designing of HCV Polymerase Inhibitors and Evaluation of the HCV Polymerase-Inhibitory Activity.

Since the active site of the HCV polymerase is the RNA replication site, a compound having the structural complementarity with the active site would inhibit the polymerase activity.

A compound having the complementarity with a site which can be a target for the inhibition of the HCV polymerase activity, for example, the RNA binding cleft, in addition to the active site, is presumed to indirectly inhibit the polymerase activity.

The inner space of the Palm domain is not involved in RNA replication, but presumably is the gap generated when the RNA is replicated. Therefore a compound having the structural complementarity with the inner space of the Palm domain is estimated to indirectly inhibit the polymerase activity.

Such three-dimensional structural information on the active site and the RNA binding cleft is important for designing/identifying HCV polymerase inhibitors by computers and such. Specifically, the complementarity of HCV polymerase inhibitors with the active site and/or RNA binding cleft, for example, the binding stability, can be compared by computers and the like means. A leading compound having the complementarity with the active site and/or the RNA binding cleft, and the derivative peripheral compounds can be rationally designed. Furthermore, in synthesis experiments, useless syntheses can be obviated, and actual evaluation of enzymes can be efficiently performed.

The complementarity with the active site and/or the RNA binding cleft can be determined, for example, by inputting the structural coordinates of the HCV polymerase and of a test compound to virtual screening programs, such as DOCK4 (UCSF), etc., using computers, and obtaining the state in which the test compound is incorporated into the active site and/or the RNA binding cleft of the HCV polymerase, as a numerical value stable in terms of conformation and energy, or as the visual model. Moreover, the complementarity of the test compound can be obtained using a part of the structural coordinate for the HCV polymerase in the same manner.

As a virtual screening program, FLEXY DOCK (Tripos) can be used in addition to DOCK4.

The structural coordinate for the test compound is available on a database for the three-dimensional structure of the chemical compounds. Alternatively the coordinate can be obtained by calculating the conformation using program software such as, Quanta (MSI), Sybyl (Tripos), Insight II (MSI), etc.

The HCV polymerase-inhibitory activity can be evaluated by comparing the thus-obtained complementarity of the test compound with the active site and/or the RNA biding cleft of the HCV polymerase.

The molecule of an inhibitor can be designed so as to have the complementarity with the active site and/or the RNA binding deft, based on the structure of the test compound. The molecules can be designed using the above program software Quanta, Sybyl, Insight II, DOCK4, FLEXY, DOCK, etc.

vi) Actual Evaluation of the HCV Polymerase-Inhibitory Activity.

The HCV polymerase-inhibitory activity can be measured by obtaining a compound having the complementarity with the active site and/or the RNA binding site of the HCV polymerase, evaluated by the above virtual screening, and contacting the obtained compound with the HCV polymerase in the presence of a template RNA and a substrate ribonucleoside triphosphate (rNTP).

The present invention enables designing or identifying HCV polymerase inhibitors by computers and such, and thus provides methodologies of rational designing of compounds and their analogues. Moreover, this invention enables efficiently evaluating the HCV polymerase-inhibitory activity, and thus provides efficient evaluation method of the HCV polymerase-inhibitory activity by the combination use of designing or identification by computers and the like means.

The present invention is illustrated in detail below with reference to the Examples, but is not construed being limited thereto.

### EXAMPLE 1

### Expression and Purification of the Native HCV Polymerase (NC5B₅₇₀)

The DNA fragment comprising the histidine tag consisting of the amino acid sequence of GSHHHHHH at the C-terminus (SEQ ID NO: 2) of NS5B was prepared by PCR using pDM22 into which cDNA of HCV-BK type virus was introduced, purchased from Research Foundation for Microbial Diseases of Osaka University, as a template, and a set of primers 5BNde1FW (SEQ ID NO: 4) and 5B570HRV (SEQ ID NO: 5). The resulting fragment was inserted into pCR2.1 vector (INVITROGEN), the sequence was confirmed, and about 1.8 kDa fragment was obtained by partial digestion with Nde1 and EcoR1.

The thus-obtained fragment was inserted into the Nde1 and EcoR1 sites in pET17b vector comprising T7 promoter (NOVAGEN), and the vector was used to transform E. coliBL21 (DE3) (NOVAGEN).

The transformants were cultured in 2×YT medium at 30° C. When OD620 reached 0.8 to 1.0, IPTG (Nacalai Tesque) was added thereto to a final concentration of 0.5 mM, and the transformants were further incubated at 30° C. for 3 hours to induce production of the target protein.

The harvested cells were disrupted with a microfluidizer and the soluble fraction was isolated and purified by subsequently performing Ni-NTA agarose (QIAGEN) column chromatography, Mono-S5/5 (PHARMACIA) column chromatography, and gel filtration with Sephacryl S-200 (PHARMACIA).

In the amino acid sequence of the obtained native HCV polymerase, methionine at the N-terminus was missing. The amino acid sequence of the obtained NS5B₅₇₀ was the amino acids 1-570 of the amino acid sequence shown in SEQ ID NO: 1, to which the histidine tag was added. In the same manner, NS5B₅₅₂, NS5B_{544,} NS5B₅₃₆, NS5B₅₃₁, and NS5B₅₉₁ were obtained using primers 5B552HRV (SEQ ID NO: 6), 5B544HRV (SEQ ID NO: 7), 5B536HRV (SEQ ID NO: 8), 5B531HRV (SEQ ID NO: 9), and 5B591HRV (SEQ ID NO: 10), respectively. The amino acid sequence of the histidine tag in the obtained NS5B₅₄₄ was GSHHDHHH.

NS5B₅₉₁ comprising the full length wild-type NS5B was purified by adding a detergent (CHAPS) and glycerol, and using the poly(U)-sepharose 4B (PHARMACIA) column chromatography in addition to the above column chromatographies.

### Expression and Purification of Selenomethionine Heavy Atom Substitution Product

In the same manner as in the expression and purification of the native HCV polymerase, the 1.8 kDa fragment obtained from pDM22 was inserted into Nde1 and EcoR1 sites of pET17b (NOVAGEN), which was used to transform E. coli B834 (DE3) (NOVAGEN).

The transformants were cultured in the medium for selenomethionine substitution mentioned below at 30° C. When OD620 reached 0.8 to 1.0, IPTG was added thereto to a final concentration of 0.5 mM, and the transformants were further cultured at 30° C. for 3 hours to induce the production of the target protein. The soluble fraction was purified in the same manner as the native HCV polymerase.

Composition of the medium for selenomethionine substitution

1. Amino acids (g/ml)

| | |
|---|---|
| Ala | 1.50 |
| Arg | 1.75 |
| Asp | 1.20 |
| Cys.HCl/H₂O | 0.10 |
| Glu | 2.00 |
| Gln | 1.00 |
| Gly | 1.63 |
| His | 0.18 |
| Ile | 0.70 |
| Leu | 0.70 |
| Lys.HCl | 1.26 |
| Phe | 0.40 |
| Pro | 0.30 |
| Ser | 6.25 |
| Thr | 0.70 |
| Tyr | 0.50 |
| Val | 0.70 |

2. Salts (g/ml)

| | |
|---|---|
| Adenosine | 1.00 |
| Guanosine | 1.33 |
| Thymine | 0.33 |
| Uracil | 1.00 |
| Succinic acid | 3.00 |
| Na Acetate.3H₂O | 1.50 |
| Ammonium-Cl | 1.50 |
| NaOH | 0.85 |
| K₂HPO₄ | 10.50 |

3. Metal, selenomethionine and others (g/ml)

| | |
|---|---|
| Mg SO₄.7H₂O | 0.25 |
| FeSO₄.7H₂O | 0.0042 |
| Glucose | 20.00 |
| Selenomethionine | 0.75 |

4. Vitamins

| | |
|---|---|
| KAO and MICHAYLUK BASAL VITAMIN solution | 10.00 ml/l |

Methionine at the N-terminus in the selenomethionine heavy atom substitution product was cleaved like in the native HCV polymerase. As a result of LC-MS, all 12 methionines were replaced by selenomethionines.

### EXAMPLE 2

Crystallization of the HCV Polymerase

The crystals of the native HCV polymerase obtained in Example 1 (NS5B₅₇₀) was prepared by vapor diffusion. Specifically, a mixture of the protein solution and a precipitation reagent solution (1:1 by volume), and a precipitation reagent solution were placed into a container that can be sealed so as not to contact the solutions with each other. The container was kept at the constant temperature for 2 to 4 weeks to obtain the crystals by vapor equilibration. The conditions for crystallization with good reproducibility are described below. To improve the rate of crystal growth in the solution, a detergent, such as n-dodecyl-β-D-maltoside, n-octanoylsucrose, and such, may be added to 0.1 to 0.3 CMC.

Conditions for crystallization of NS5B₅₇₀

Protein solution: NS5B₅₇₀ dissolved in 5 mM dithiothreitol (DTT) solution to 10±5 mg/ml.
Precipitation reagent solution: containing 21 to 28% (w/v) polyethylene glycol 4000, 0.2 to 0.35 M ammonium acetate, 0.1 M sodium acetate, 0.02 M TES buffer (N-tris(hydroxy-methyl)methyl-2-aminoethanesulfonic acid, pH 6.0 to 7.5).
Crystallization temperature: 22±2° C.
   Conditions for crystallization of NS5B₅₄₄
Protein solution: NS5B₅₄₄ dissolved in 5 mM dithiothreitol (DTT) solution to 10±3 mg/ml.
Precipitation reagent solution: the solution containing 2.0 to 5.0% (w/v) polyethylene glycol 8000, 5% (v/v) isopropanol, 0.1 M sodium citrate buffer (pH 5.5 to 6.5).
Crystallization temperature: 4±2° C.

The crystals of NS5B₅₄₄, NS5B₅₃₆, and NS5B₅₃₁ were obtained in the same manner.

Table 1 shows the crystallographical parameters for NS5B₅₇₀ and NS5B₅₄₄. The crystallographical parameters for NS5B₅₃₆ and NS5B₅₃₁ were similar to those for NS5B₅₄₄.

TABLE 1
Crystallographic parameters

| | | | Number of |
|---|---|---|---|
| | | | independent molecule |
| Crystal | Space group | Lattice constants | in asymmetric unit |
| NS5B₅₇₀ | P4₃2₁2 | a = b = 63.7(±0.7) Å | 1 |
| | | c = 262.9(±3.0)Å | |
| | | α = β = γ = 90° | |
| NS5B₅₄₄ | P2₁2₁2 | a = 67.6(±0.7)Å | 1 |
| | | b = 95.9(±1.0)Å | |
| | | c = 97.6(±1.0)Å | |
| | | α = β = γ = 90° | |

### EXAMPLE 3

### Crystal Structure Analysis of the HCV Polymerase

The crystals of the native HCV polymerase (NS5B₅₇₀) were soaked in the precipitation reagent solution used in the crystallization of NS5B₅₇₀ described above containing heavy atoms, such as platinum, uranium, or osmium, to obtain the heavy atom substitution products.

The selenomethionine HCV polymerase was crystallized by vapor diffusion in the same manner described above.

Diffraction intensity of the obtained platinum heavy atom substitution product, uranium heavy atom substitution product, and osmium heavy atom substitution product of the native HCV polymerase crystal, the native polymerase crystal, as well as the selenomethionine HCV polymerase crystal were measured using Raxis IIc (Rigaku), and BL6B of synchrotron facility KEK-PF, and BL45XU of SPring-8.

The X-ray diffraction data for the platinum heavy atom substitution product, the uranium heavy atom substitution product, and the osmium heavy atom substitution product were processed with DENSO (HKL) and SCALA, FHSCAL, SCALEIT of CCP4 program (Council for the Central Laboratory of the Research Councils). The scale of data was adjusted to that of the diffraction intensity of the native HCV polymerase (NS5B₅₇₀) crystal so that the diffraction intensity of the products could be compared with each other. The first locations of the heavy atoms were determined processing the data of the uranium heavy atom substitution product, the osmium heavy atom substitution product, and the native HCV polymerase (NS5B₅₇₀) crystal with the program software Shelx (Professor Sheldrick; Crystallographic Computing 3, Clarendon Press, Oxford 184-189 (1985)). Subsequently, the accurate locations of each heavy atom were determined using the program software MLPHARE in CCP4 and SHARP (Laboratory of Molecular Biology) to calculate the initial phase angles. The improvement of the initial phase and expansion of the phase within 2.5 Å were calculated using the program software DM in CCP4 to prepare the Fourier map.

The selenomethionine HCV polymerase (NS5B₅₇₀) corresponds to the amino acid sequence of SEQ ID NO: 1 in which 12 methionine residues are replaced with selenomethionines. The differential Fourier map was prepared for this selenomethionine HCV polymerase using the phase information described above. The differential Fourier map of the diffraction data measured at the X-ray wave length λ=1.0400 Å, and the diffraction intensity data measured at λ=0.9797 Å, in which 11 peaks corresponding to a selenium atom were confirmed, was used as a guide for the structure determination.

The structure of the HCV polymerase was determined based on the obtained Fourier map using the program software O (DatOno AB).

Refinement was performed using torsion angle or maximum likelihood refinement of the program software X-PLOR98 (MSI). Ramachandran plot obtained by using the program software PROCHECK (J. Appl. Cryst. 26, 283-290, 1993) confirmed that there was no amino acid residue with unacceptable structure. The structural coordinates were shown in Table 2. Each symbol in Tables means as follows.

(Atom type: from the left, the serial numbers of atoms contained in the coordinates, types of atoms and location of the atoms in amino acids, numbers: amino acid residue number of amino acids comprising the atoms, X, Y, and Z: coordinates of the atoms, Occ: occupancy, B: temperature factor)

The structural coordinate of NS5B₅₄₄, in NS5B₅₄₄, NS5B₅₃₆ and NS5B₅₃₁ obtained by the molecular replacement method was shown in Table 3. The structural coordinates of NS5B₅₃₆ and NS5B₅₃₁ were similar to that shown in Table 3.

LENGTHY TABLE REFERENCED HERE
US20070042353A1-20070222-T00001
Please refer to the end of the specification for access instructions.

LENGTHY TABLE REFERENCED HERE
US20070042353A1-20070222-T00002
Please refer to the end of the specification for access instructions.

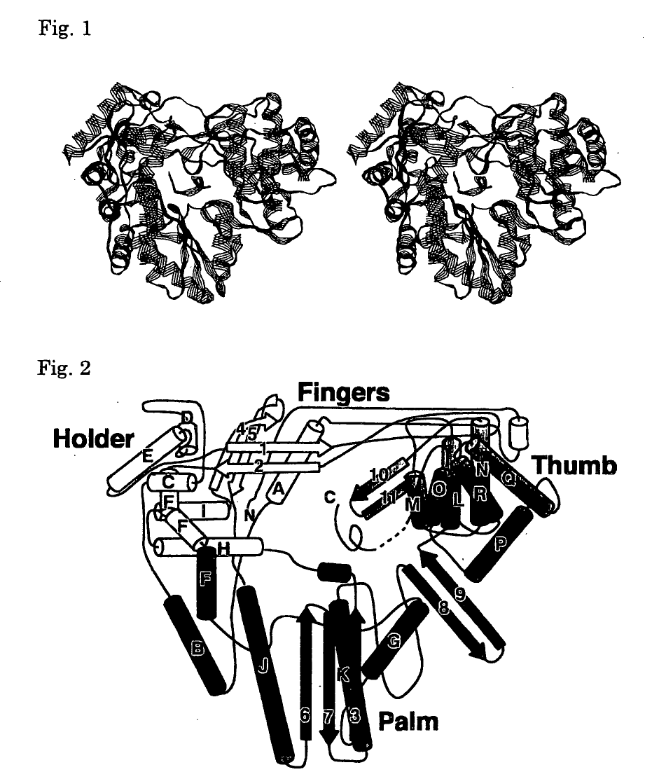

The structure of the HCV polymerase was visualized using the program software RasMol (Free Soft, Roger Sayle, Glaxo Research & Development, Greenford, Middlesex, UK). FIG. 1 shows the three-dimensional structure obtained by visualizing the structural coordinate of NS5B₅₇₀ in Table 2.

Crystal structure analysis revealed that the crystal of HCV polymerase NS5B₅₇₀ belongs to the space group of P4₃2₁2 of a=b=63.7 Å, c=262.9 Å, and the HCV polymerase is 67×63×68 Å spherical protein comprising the cone shape in the structure.

The three-dimensional structure of the HCV polymerase NS5B₅₇0 characteristically has a glove-like structure shown in FIG. 1, comprising Fingers, Palm, Thumb, and Holder domains. FIG. 2 schematically shows the structure of the HCV polymerase.

The Fingers domain comprises four β sheets and one a helix, similar to the structure of the HIV reverse transcriptase, although there is no similarity in the amino acid sequence to this enzyme. There are two long loops (one loop extending from the N-terminus to a A helix, and the other loop between β1 and β2), and a net is formed from the lower part of the cone shape to the upper end of the Thumb domain. The lower part of the net is open, and presumably is the entrance for a substrate ribonucleoside triphosphate (rNTP).

It is known that the structure of the poliovirus polymerase (Structure 5, 1109-1122, 1997) comprises Fingers, Palm, and Thumb domains. The structure of the Fingers domain is disordered except for the net end containing a short helix in which the Fingers domain extends to the Thumb domain. The region corresponding to the connecting region between the Holder and Palm domains in the HCV polymerase was identified as the Fingers domain, however, most of the rest of the structure in the Fingers domain has not been revealed yet.

The Holder domain consists of two helixes, αH and αI, located like supporting this region, a part of each αC, αD, αE, and αF, and a long loop like inserted to the Fingers and Palm domains between αD and αE. This domain forms a valley which is one wall of the cone shape between the Palm domain and this domain, and the U-shaped valley between the Fingers domain and the domain. In two valleys, basic amino acid residue align, which are positively charged. The positively charged surface conveniently binds to a negatively charged template RNA, thus the U-shaped valley is supposed to be an entrance for a template RNA.

The Palm domain comprises the structure similar to HIV reverse transcriptase, E. coli, or Taq DNA-dependent DNA polymerase and T7 DNA-dependent polymerase.

The Thumb domain consists of seven helixes, two distorted β sheets. The core structure of this domain comprises the structure similar to the HIV reverse transcriptase. The β sheet extending from the apex of the Thumb domain consists of nonhydrophilic residues, except for the hydrophilic junction, and hangs down to the center of the cone shape, like pushing the C-terminal nonhydrophilic region. This long β sheet is not observed in the other polymerases.

The N-terminus of the HCV polymerase forms mimic β sheet at the center of the Fingers domain with β5. This means that N-terminus-truncated variants lose the replicase activity.

### EXAMPLE 4

### Determination of the Active Site and the RNA Binding Cleft in the HCV Polymerase

The active site and the RNA binding cleft in the HCV polymerase were determined based on the three-dimensional structures of the obtained HCV polymerases (NS5B₅₇₀, NS5B₅₄₄, NS5B₅₃₆, and NS5B₅₃₁), the conformational variation among each HCV polymerase, and the comparison of the three-dimensional structures with other proteins comprising the similar functions.

The Palm domain of the HCV polymerase was revealed to have the structure similar to HIV reverse transcriptase, E. coli, or Taq DNA-dependent DNA polymerase, and T7 DNA-dependent polymerase. Comparison of the conserved amino acids sequences between the active site of the structurally known Palm domains of the other polymerase and the Palm domain of the HCV polymerase deduced that the active site is the space formed by Asp 220, 318, and 319, Lys 141, Arg 158, and/or the hydrophilic shallow cavity formed by Ser 282, Thr 287, and Asn 291.

Asp 225 corresponds to Tyr 115 of the HIV reverse tranascriptase and this difference of the amino acids presumably determines whether the substrate is rNTP or dNTP. Arg 158 and Lys 141 of the Fingers domain, the conserved residues between the HCV polymerase and the HIV reverse transcriptase, would have an important role in the binding of rNTP.

The Thumb domain of the HCV polymerase can structurally move against the Palm and Fingers domains and this movement changes the inner space of the Palm domain. This movement can be compared to the open and closed states of a glove. This space was confirmed to be formed by the regions of amino acids 197 to 223, 310 to 325, and 348 to 366, and is designated “the inner space of the Palm domain”.

A compound existing in this space presumably inhibits the spatial formation and inactivates the polymerase activity. It is rationally assumed that “the inner space of the Palm domain” can be a target for HCV polymerase inhibitors. Even if distortion is generated to some extent, this space is a part of the RNA binding site, and this region may thus shift in about 1 to about 20, preferably about 1 to about 10, and more preferably about 1 to about 5 amino acids.

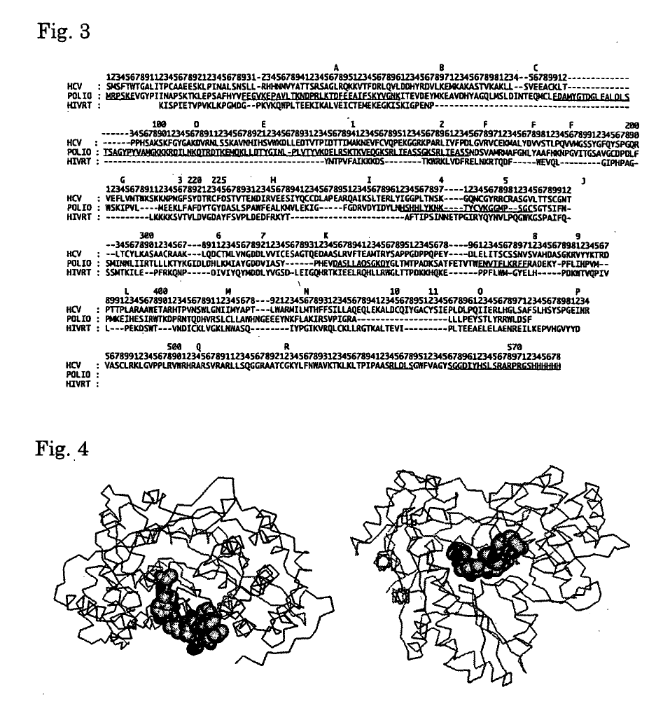

FIG. 3 compares the amino acid sequences of the HCV polymerase, poliovirus polymerase, and HIV reverse transciptase. HCV, POLIO, and HIVRT in the figure indicate HCV polymerase, poliovirus polymerase, and HIV reverse transcriptase, respectively. The underlined sequences indicate the parts where the structures have not been clarified by the above structure analysis. It is difficult to deduce the active site and RNA binding cleft only from the two-dimensional structures, as obvious in these amino acid sequences.

The three-dimensional structure analysis of NS5B₅₇₀ showed that the C-terminal structure at positions 545 to 570 can be incorporated into a part of the RNA binding cleft of the HCV polymerase itself. FIG. 4 schematically shows the structure of NS5B₅₇₀, emphasizing its C-terminus. The RNA binding cleft in which the C-terminal structure is incorporated is narrow in comparison with that the C-terminus-truncated variants, NS5B₅₄₄ NS5B₅₃₆, and NS5B₅₃₁, and the glove is slightly closed. This supports that the RNA binding cleft can be a target for the inhibition of the polymerase activity.

Lys 90, 98, 106, and 175, and Arg 168 in the Holder domain, and Arg 465 in the Thumb domain are located within 5 Å from the phosphodiester backbone of the RNA double strand model in the binding model made on the computer, consisting of the tentative short double strand model formed by a template and a primer. These amino acids would have an important role on the binding of the RNA strand. Therefore, these regions can also be a target for the inhibition of the polymerase activity.

### EXAMPLE 5

Evaluation of the HCV Polymerase Activity

Synthesis of a Template RNA

The DNA fragment (148 bp) containing polyU and 3′ X sequence was completely synthesized using synthetic primers designed based on the sequence of the 3′ untranslational region of the HCV genome, and cloned into plasmid pBluescript SK II (+) (Stratagene). The cDNA encoding the full length NS5B prepared in the same manner as in Example 1 was digested with restriction enzyme KpnI to obtain the cDNA fragment covers the region from the restriction enzyme cleavage site to the stop codon. This cDNA fragment was ligated to the upstream of 3′ untranslational region DNA of pBluescript SK II (+). The DNA insert of about 450 bp in total was used as a template for preparing the template RNA. The plasmid was cleaved just after 3′ X sequence, circulated, treated with phenol/chloroform, and purified by ethanol precipitation to recover the DNA.

Using the purified DNA as a template, utilizing the promoter in pBluescript SK II (+), RNA was synthesized by the run-off method using MEGAscript RNA synthesis kit (Ambion), and T7 RNA polymerase (at 37° C. for 3 hours). DNaseI was added thereto and further incubated for 1 hour, and the template DNA was decomposed and removed to obtain the crude RNA product. The crude product was treated with phenol/chloroform, and purified by ethanol precipitation to obtain the desired template RNA. After confirming the quality of the RNA by agarose gel electrophoresis, the RNA was stored at −80° C. The RNA is a template suitable for the highly sensitive measurement of the polymerase activity.

Measurement of the HCV Polymerase Activity

The reaction mixture with the composition mentioned below (30 μl) was reacted at 25° C. for 90 min.
Reaction mixture: the HCV polymerase obtained in Example 1 (1 μg/ml), the template RNA obtained in Example 5 (10 μg/ml), ATP (50 μM), GTP (50 μM), CTP (50 μM), UTP (2 μM), [5, 6-³H] UTP (46 Ci/mmol (Amersham), 1.5 μCi), 20 mM Tris-HCl (pH 7.5), EDTA (1 mM), MgCl₂ (5 mM), NaCl (50 mM), DTT (1 mM), BSA (0.01%).

The reaction was terminated by adding 10% trichloroacetic acid at 4° C. and 1% sodium diphosphate solution (150 μl) to the reaction mixture, and kept in ice for 15 min to precipitate the RNA. The RNA was trapped on a glass filter (Whatman, GF/C) by suction filtration. The filter was washed with a solution containing 1% trichloroacetic acid and 0.1% sodium diphosphate, then with 90% ethanol, and dried. A liquid scintillation cocktail (Packard) was added to the RNA synthesized by the enzyme reaction, and the radioactivity of the RNA was measured by a liquid scintillation counter.

The activity of the HCV polymerase was calculated from the value of radioactivity in the enzyme reaction of each NS5B using the radioactivity in the enzyme reaction of NS5B₅₉₁ as a standard. Table 4 shows the result.

TABLE 4
HCV polymerase activity

| | HCV polymerase | | | | | |
|---|---|---|---|---|---|---|
| | NS5B₅₉₁ | NS5B₅₇₀ | NS5B₅₅₂ | NS5B₅₄₄ | NS5B₅₃₆ | NS5B₅₃₁ |
| relative | 100 | 3 | 147 | 2014 | 2107 | 2149 |
| activity | | | | | | |
| (%) | | | | | | |

### EXAMPLE 6

HCV Polymerase Useful for Measuring the HCV Polymerase Activity

The above results confirmed that NS5B₅₄₄, NS5B₅₃₆, and NS5B₅₃₁, show the HCV polymerase activity higher than the wild-type HCV polymerase NS5B₅₉₁, and NS5B₅₇₀; surprisingly 20-fold or more higher than NS5B₅₉₁.

Crystal structural analysis confirmed that evaluation of the HCV polymerase activity for the various mutans showed that NS5B₅₅₂ did not have high activity, and amino acid residues 517 to 526 of NS5B is the helix structure contained in the secondary structure which has an important role for maintaining higher structure (Example 3). From this result, the HCV polymerase comprising the amino acid sequence of NS5B₅₂₆, NS5B₅₂₇, NS5B₅₂₈, . . . to NS5B₅₅₁ are assumed to comprise high HCV polymerase activity.

These NS5B₅₄₄, NS5B₅₃₆ and NS5B₅₃₁ comprising the high HCV polymerase activity are extremely useful for evaluating the inhibition level of the inhibitor candidate compounds in the evaluation of inhibitory activity in vitro, and HCV polymerase inhibitors can be efficiently designed or identified.

In addition, the inhibitors can be efficiently designed or identified by the above virtual screening based on structural coordinates of the HCV polymerase, for example, the structural coordinate of NS5B₅₄₄ shown in Table 3.

### EXAMPLE 7

### Identification of Inhibitors for the HCV Polymerase

The three-dimensional structural analysis of NS5B₅₇₀ obtained in Example 3 revealed that the polypeptide region at positions 545 to 570, the C-terminal structure of the HCV polymerase NS5B₅₇₀, is incorporated into a part of the RNA binding cleft of the HCV polymerase itself. Moreover, the comparison with the three-dimensional structure of NS5B₅₄₄ or other variants, confirmed that the RNA binding cleft in which the C-terminal structure is incorporated becomes slightly narrower than that in the C-terminus-truncated variants, NS5B₅₄₄, NS5B₅₃₆, and NS5B₅₃₁. This is compared to the slightly closed state of the glove. This suggests that the RNA binding cleft can be a target for the for polymerase inhibitors.

On the other hand, the measurement of the HCV polymerase activity in Example 5 revealed that the HCV polymerase activities of NS5B₅₄₄, NS5B₅₃₆, and NS5B₅₃₁ were higher than those of NS5B₅₉₁, NS5B₅₇₀, and NS5B₅₅₂.

These facts suggest that the polypeptide regions at positions 545 to 570 and a compound with the similar structure to the region can be an HCV polymerase inhibitor.

It is easily assumed that the polypeptide fragment at positions 545 to 570 can be an HCV polymerase inhibitor.

The difference of the HCV polymerase activities was observed between NS5B₅₅₂ and NS5B₅₄₄. This indicates that the polypeptide fragment at positions 545 to 552 and its partial fragment or a compound containing these fragments are particularly effective as HCV polymerase inhibitors.

The computational analysis of the three-dimensional structure using the program software QUANTA (MSI) confirmed that the region at positions 545 to 552 maintains hydrophobic interaction with the region comprising the hydrophobic surface existing in “boundary site between Thumb and Palm domains.” Furthermore, the computational analysis confirmed that in the polypeptide fragment at positions 545 to 552, Leu 547, Trp 550, and Phe 551, especially Trp 550 and Phe 551, strongly interact with said hydrophobic surface.

Specifically “boundary site between Thumb and Palm domains” means the site which is formed by Ser 196, Pro 197, Ile 413, Met 414, Ile 447, Tyr 448, Tyr 452, Ile 454, Ile 462, and Leu 466 in the amino acid sequence of the HCV polymerase. This experimental result suggests “boundary domain between Thumb and Palm domains” or a domain containing a part of it can be a target for the HCV polymerase inhibitors. Particularly, it was confirmed that the polypeptide region at positions 545 to 552 and its partial fragment or a compound containing these fragments are effective as HCV polymerase inhibitors.

Next, a synthetic peptide was prepared by a conventional method and its HCV polymerase-inhibitory activity was assessed. The synthetic peptide consists of the polypeptide region at positions 546 to 551 of HCV polymerase NS5B to both ends of which a Lys residue is attached and represented by the formula Lys-Asp-Leu-Ser-Gly-Trp-Phe-Lys. The synthetic peptide and NS5B₅₄₄ were pre-incubated at 25° C. for 30 minutes, and the polymerase activity was measured in the same manner as in Example 5. The synthetic peptide inhibited the polymerase activity 40 to 50% at a final concentration of 30 μM.

LENGTHY TABLE
The patent application contains a lengthy table section. A copy of the table is available in electronic form from the USPTO web site (http://seqdata.uspto.gov/?pageRequest=docDetail&DocID=US20070042353A1). An electronic copy of the table will also be available from the USPTO upon request and payment of the fee set forth in 37 CFR 1.19(b)(3).

What is claimed is:

1. A polypeptide derived from HCV polymerase NS5B having an HCV polymerase activity and consisting of an amino acid sequence X-Y, wherein X is a consecutive amino acid sequence which is a portion of the NS5B, an N-terminal amino acid of X is the amino acid residue 1 (Ser) of the NS5B, and a C-terminal amino acid residue of X is any one of amino acid residues 531 (Lys) to 570 (Arg) of the NS5B; and wherein Y is a carboxyl group or an amino acid sequence which is not derived from NS5B; and one or more amino acids in the amino acid sequence of X may be modified, and methionine residues in the amino acid sequence of X may be replaced by selenomethionoine residues.

2. The polypeptide of claim 1, wherein the C-terminal amino acid residue of X is any one of amino acid residues 536 (Leu) to 552 (Val) of the NS5B.

3. The polypeptide of claim 2, wherein the C-terminal amino acid residue of X is any one of amino acid residues 536 (Leu) to 544 (Gln) of the NS5B.

4. The polypeptide of claim 2, wherein the C-terminal amino acid residue of X is any one of amino acid residues 531 (Lys) to 544 (Gln) in the NS5B.

5. The polypeptides of any one of claims 1 to 4, wherein methionine residues in the amino acid sequence of X are replaced by selenomethionine residues.

6. The polypeptides of any one of claims 1 to 5, wherein Y is an amino acid sequence not derived from NS5B, and said amino acid sequence is suitable for a column purification.

7. The polypeptides of any one of claims 1 to 6, wherein the NS5B comprises an amino acid sequence of SEQ ID NO: 1.

8. The polypeptide of claim 1, wherein said polypeptide is identified by an three-dimensional structural coordinates shown in Table 2 or 3.

9. A crystal comprising the polypeptide of any one of claims 1 to 8.

10. A DNA encoding the polypeptide of any one of claims 1 to 8.

11. A method for determining a three-dimensional structural coordinates of a cocomplex or a variant of HCV polymerase NS5B by the molecular replacement method using a three-dimensional structure coordinate of said NS5B.

12. A method for designing or identifying HCV polymerase inhibitors, which comprises determining the complementarity of a test compound with an active site and/or RNA binding cleft of a polypeptide using the three-dimensional structural coordinate of said polypeptide or its part and the three dimensional structural coordinate of the test compound, wherein said polypeptide is derived from the HCV polymerase NS5B having an HCV polymerase activity and consisting of an amino acid sequence X-Y, wherein X is a consecutive amino acid sequence which is a portion of the NS5B, an N-terminal amino acid of X is the amino acid residue 1 (Ser) of the NS5B, a C-terminal amino acid residue of X is any one of amino acid residues 531 (Lys) to 570 (Arg) of the NS5B: and wherein Y is a carboxyl group or another amino acid sequence which is not derived from NS5B; and one or more amino acids in X may be modified, and methionine residues in the amino acid sequence of X may be replaced by selenomethionine residues.

13. A method for designing or identifying HCV polymerase inhibitors, which comprises the steps of:
(a) determining the complementarity of a test compound with an active site and/or RNA binding cleft of the a polypeptide using a three-dimensional structural coordinate of said polypeptide or its part and a three-dimensional structural coordinate of said test compound, wherein said polypeptide is derived from the HCV polymerase NS5B having an HCV polymerase activity and consisting of an amino acid sequence X-Y, wherein X is a consecutive amino acid sequence which is a portion of the NS5B, an N-terminal amino acid of X is the amino acid residue 1 (Ser) of the NS5B, a C-terminal amino acid residue of X is any one of amino acid residues 531 (Lys) to 570 (Arg) of the NS5B; and wherein Y is a carboxyl group of another amino acid sequence which is not derived from NS5B; and one or more amino acids in X may be modified, and methionine residues in the amino acid sequence of X may be replaced by selenomethionin residues;
(b) determining HCV polymerase-inhibitory activity of said test compound; and
(c) designing or determining HCV polymerase inhibitors using the complementarity data of said test compound determined in the above (a), and the inhibitory activity data obtained in the above (b).

14. The method of any one of claims 11 to 13, wherein the three-dimensional structural coordinate of the polypeptide is any one of the three-dimensional structural coordinates shown in Table 2 or 3.

15. A method for identifying HCV polymerase inhibitors, which comprises the steps of:
(a) obtaining a polypeptide, which is derived from the HCV polymerase NS5B has an HCV polymerase activity, and consisting of the amino acid sequence X′-Y, wherein X′ is a consecutive amino acid sequence which is a portion of the NS5B, an N-terminal amino acid of X′ is the amino acid residue 1 (Ser) of the NS5B, a C-terminal amino acid residue of X′ is any one of amino acid residues 531 (Lys) to 544 (Gln) of the NS5B; and wherein Y is a carboxyl group or another amino acid sequence which is not derived from NS5B; and one or more amino acids in X′ may be modified, and methionine residues in the amino acid sequence of X′ may be replaced by selenomethionin residues;
(b) determining the HCV polymerase activity of said polypeptide by reacting said polypeptide obtained in the above (a) with a template RNA and substrates in the presence of a test compound;
(c) determining the HCV polymerase activity of said polypeptide by reacting polypeptide obtained in the above (a) with a template RNA and substrates in the absence of said test compound; and
(d) comparing the HCV polymerase activity of the above (b) with the HCV polymerase activity of the above (c).

16. An HCV polymerase inhibitor, identified by the method in any one of claims 12 to 15.

17. An HCV polymerase inhibitor that inhibits the HCV polymerase activity of HCV polymerase NS5B by acting the boundary between the Thumb and Palm domains of NS5B.

18. The HCV polymerase inhibitor of claim 17, wherein said inhibitor is a polypeptide represented by the formula (I) or a pharmaceutically acceptable salf thereof:
Z¹-Z²-Z³-Leu-Z⁴-Z⁵-Trp-Phe-Z⁶ (I)
wherein Z¹ and Z⁶ each represent a hydrophilic group or an amino acid residue; Z² and Z³ each represent a single bond or an amino acid residue; and Z⁴ and Z⁶ each represent an amino acid residue.


SEQUENCE LISTING (28 sequences)

SEQ ID NO: 1 - protein, 591 aa, Hepatitis C virus
SMSYTWTGALITPCAAEESKLPINALSNSLLRHHNMVYATTSRSAGLRQKKVTFDRLQVLDDHYRDVLKEMKAKASTVKAKLLSVEEACKLTPPHSAKSKFGYGAKDVRNLSSKAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPRWFMLCLLLLSVGVGIYLLPNR

SEQ ID NO: 2 - DNA, 1743 bp, Artificial Sequence
  Description of Artificial Sequence DNA encoding fusion protein consisting of a portion of HCV polymerase and histidine tag at the C-terminus
  CDS (1)..(1743)
atgtcaatgtcctacacatggacaggcgccttgatcacgccatgcgctgcggaggaaagcaagctgcccatcaacgcgttgagcaactctttgctgcgccaccataacatggtttatgccacaacatctcgcagcgcaggcctgcggcagaagaaggtcacctttgacagactgcaagtcctggacgaccactaccgggacgtgctcaaggagatgaaggcgaaggcgtccacagttaaggctaaactcctatccgtagaggaagcctgcaagctgacgcccccacattcggccaaatccaagtttggctatggggcaaaggacgtccggaacctatccagcaaggccgttaaccacatccactccgtgtggaaggacttgctggaagacactgtgacaccaattgacaccaccatcatggcaaaaaatgaggttttctgtgtccaaccagagaaaggaggccgtaagccagcccgccttatcgtattcccagatctgggagtccgtgtatgcgagaagatggccctctatgatgtggtctccacccttcctcaggtcgtgatgggctcctcatacggattccagtactctcctgggcagcgagtcgagttcctggtgaatacctggaaatcaaagaaaaaccccatgggcttttcatatgacactcgctgtttcgactcaacggtcaccgagaacgacatccgtgttgaggagtcaatttaccaatgttgtgacttggcccccgaagccagacaggccataaaatcgctcacagagcggctttatatcgggggtcctctgactaattcaaaagggcagaactgcggttatcgccggtgccgcgcgagcggcgtgctgacgactagctgcggtaacaccctcacatgttacttgaaggcctctgcagcctgtcgagctgcgaagctccaggactgcacgatgctcgtgaacggagacgacctcgtcgttatctgtgaaagcgcgggaacccaagaggacgcggcgagcctacgagtcttcacggaggctatgactaggtactccgccccccccggggacccgccccaaccagaatacgacttggagctgataacatcatgttcctccaatgtgtcggtcgcccacgatgcatcaggcaaaagggtgtactacctcacccgtgatcccaccacccccctcgcacgggctgcgtgggagacagctagacacactccagttaactcctggctaggcaacattattatgtatgcgcccactttgtgggcaaggatgattctgatgactcacttcttctccatccttctagcgcaggagcaacttgaaaaagccctggactgccagatctacggggcctgttactccattgagccacttgacctacctcagatcattgaacgactccatggccttagcgcattttcactccatagttactctccaggtgagatcaatagggtggcttcatgcctcaggaaacttggggtaccacccttgcgagtctggagacatcgggccaggagcgtccgcgctaggctactgtcccagggggggagggccgccacttgtggcaagtacctcttcaactgggcagtgaagaccaaactcaaactcactccaatcccggctgcgtcccagctggacttgtccggctggttcgttgctggttacagcgggggagacatatatcacagcctgtctcgtgcccgaccccgcggatcccatcaccatcaccatcactaataa

SEQ ID NO: 3 - protein, 579 aa, Artificial Sequence
  Description of Artificial Sequence Fusion protein consisting of a portion of HCV polymerase and histidine tag at the C-terminus
MSMSYTWTGALITPCAAEESKLPINALSNSLLRHHNMVYATTSRSAGLRQKKVTFDRLQVLDDHYRDVLKEMKAKASTVKAKLLSVEEACKLTPPHSAKSKFGYGAKDVRNLSSKAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPRGSHHHHHH

SEQ ID NO: 4 - DNA, 30 bp, Artificial Sequence
  Description of Artificial Sequence Synthetic primer, 5BNde1FW
  primer_bind (1)..(30)
catatgtcaatgtcctacacatggacagcc

SEQ ID NO: 5 - DNA, 57 bp, Artificial Sequence
  Description of Artificial Sequence Synthetic primer, 5B570HRV
  primer_bind (1)..(57)
ttattagtgatggtgatggtgatgggatccgcggggtcgggcacgagacaggctgtg

SEQ ID NO: 6 - DNA, 57 bp, Artificial Sequence
  Description of Artificial Sequence Synthetic primer, 5B552HRV
  primer_bind (1)..(57)
ttattagtgatggtgatggtgatgggatccaacgaaccagccggacaagtccagctg

SEQ ID NO: 7 - DNA, 57 bp, Artificial Sequence
  Description of Artificial Sequence Synthetic primer, 5B544HRV
  primer_bind (1)..(57)
ttattagtgatggtgatggtgatgggatccctgggacgcagccgggattggagtgag

SEQ ID NO: 8 - DNA, 67 bp, Artificial Sequence
  Description of Artificial Sequence Synthetic primer, 5B536HRV
  primer_bind (1)..(67)
ttattagtgatggtgatggtgatgggatccgagtttgagtttggtcttcactgcccagttgaagagg

SEQ ID NO: 9 - DNA, 60 bp, Artificial Sequence
  Description of Artificial Sequence Synthetic primer, 5B531HRV
  primer_bind (1)..(60)
ttattagtgatggtgatggtgatgggatcccttcactgcccagttgaagaggtacttgcc

SEQ ID NO: 10 - DNA, 52 bp, Artificial Sequence
  Description of Artificial Sequence Synthetic primer, 5B591HRV
  primer_bind (1)..(52)
ttattaatggtgatggtgatggtgtccggatcgattggggagcaggtagatg

SEQ ID NO: 11 - protein, 9 aa, Hepatitis C virus
  MOD_RES (1)..(3): Variable amino acid or not present
  MOD_RES (5)..(6): Variable amino acid
  MOD_RES (9): Variable amino acid or not present
XXXLXXWFX

SEQ ID NO: 12 - protein, 8 aa, Hepatitis C virus
KDLSGWFK

SEQ ID NO: 13 - protein, 9 aa, Hepatitis C virus
KKDLSGWFK

SEQ ID NO: 14 - protein, 8 aa, Hepatitis C virus
KDLSGWFV

SEQ ID NO: 15 - protein, 8 aa, Hepatitis C virus
LDLSGWFK

SEQ ID NO: 16 - protein, 8 aa, Hepatitis C virus
LDLSGWFV

SEQ ID NO: 17 - protein, 7 aa, Hepatitis C virus
DLSGWFV

SEQ ID NO: 18 - protein, 6 aa, Hepatitis C virus
DLSGWF

SEQ ID NO: 19 - protein, 6 aa, Hepatitis C virus
LSGWFV

SEQ ID NO: 20 - protein, 5 aa, Hepatitis C virus
LSGWF

SEQ ID NO: 21 - protein, 6 aa, Hepatitis C virus
LSGWFK

SEQ ID NO: 22 - protein, 6 aa, Hepatitis C virus
KLSGWF

SEQ ID NO: 23 - protein, 5 aa, Hepatitis C virus
LGGWF

SEQ ID NO: 24 - protein, 5 aa, Hepatitis C virus
LSDWF

SEQ ID NO: 25 - protein, 8 aa, Artificial Sequence
  Description of Artificial Sequence Synthetic peptide
GSHHDHHH

SEQ ID NO: 26 - protein, 578 aa, Hepatitis C virus
SMSFTWTGALITPCAAEESKLPINALSNSLLRHHNMVYATTSRSAGLRQKKVTFDRLQVLDDHYRDVLKEMKAKASTVKAKLLSVEEACKLTPPHSAKSKFGYGAKDVRNLSSKAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYKTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASRLDLSGWFVAGYSGGDIYHSLSRARPRGSHHHHHH

SEQ ID NO: 27 - protein, 465 aa, Human poliovirus 1
MRPSKEVGYPIINAPSKTKLEPSAFHYVFEGVKEPAVLTKNDPRLKTDFEEAIFSKYVGNKITEVDEYMKEAVDHYAGQLMSLDINTEQMCLEDAMYGTDGLEALDLSTSAGYPYVAMGKKKRDILNKQTRDTKEMQKLLDTYGINLPLVTYVKDELRSKTKVEQGKSRLIEASSGKSRLIEASSNDSVAMRMAFGNLYAAFHKNPGVITGSAVGCDPDLFWSKIPVLMEEKLFAFDYTGYDASLSPAWFEALKMVLEKIGFGDRVDYIDYLNHSHHLYKNKTYCVKGGMPSGCSGTSIFNSMINNLIIRTLLLKTYKGIDLDHLKMIAYGDDVIASYPHEVDASLLAQSGKDYGLTMTPADKSATFETVTWENVTFLKRFFRADEKYPFLIHPVMPMKEIHESIRWTKDPRNTQDHVRSLCLLAWHNGEEEYNKFLAKIRSVPIGRALLLPEYSTLYRRWLDSF

SEQ ID NO: 28 - protein, 320 aa, Human immunodeficiency virus
KISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDICKLVGKLNWASQIYPGIKVRQLCKLLRGTKALTEVIPLTEEAELELAENREILKEPVHGVYYD